(19) United States
(12) Patent Application Publication
(10) Pub. No.: US 2012/0259367 A1
(43) Pub. Date: Oct. 11, 2012

(54) LUMBAR-SACRAL IMPLANT ALLOWING VARIABLE ANGLE FIXATION

(75) Inventor: Eric C. Lange, Pleasanton, CA (US)

(73) Assignee: Kyphon SARL, Neuchatel (CH)

(21) Appl. No.: 13/082,966

(22) Filed: Apr. 8, 2011

(51) Int. Cl. A61B 17/70; A61B 17/88

(52) U.S. Cl. 606/249; 606/279

(57) ABSTRACT

Medical devices for the treatment of spinal conditions are described herein. The medical device includes a main body that is adapted to be placed between the L5 vertebra and the sacrum so that the main body acts as a spacer with respect to the L5 vertebra and the sacrum to maintain distraction therebetween when the spine moves in extension. Channels are formed in the lower portion of the main body and allow a fixation device to extend through each channel at different angles. A locking mechanism is disposed in the channels to lock the fixation devices in each channel with respect to the device in a desired orientation.

### BACKGROUND

This invention relates generally to devices for the treatment of spinal conditions, and more particularly, to the treatment of various spinal conditions that cause back pain. Even more particularly, this invention relates to devices that may be placed between adjacent spinous processes to treat various spinal conditions. For example, spinal conditions that may be treated with these devices may include spinal stenosis, degenerative disc disease (DDD), disc herniations and spinal instability, among others.

The clinical syndrome of neurogenic intermittent claudication due to lumbar spinal stenosis is a frequent source of pain in the lower back and extremities, leading to impaired walking, and causing other forms of disability in the elderly. Although the incidence and prevalence of symptomatic lumbar spinal stenosis have not been established, this condition is the most frequent indication of spinal surgery in patients older than 65 years of age.

Lumbar spinal stenosis is a condition of the spine characterized by a narrowing of the lumbar spinal canal. With spinal stenosis, the spinal canal narrows and pinches the spinal cord and nerves, causing pain in the back and legs. It is estimated that approximately 5 in 10,000 people develop lumbar spinal stenosis each year. For patients who seek the aid of a physician for back pain, approximately 12%-15% are diagnosed as having lumbar spinal stenosis.

Common treatments for lumbar spinal stenosis include physical therapy (including changes in posture), medication, and occasionally surgery. Changes in posture and physical therapy may be effective in flexing the spine to decompress and enlarge the space available to the spinal cord and nerves—thus relieving pressure on pinched nerves. Medications such as NSAIDS and other anti-inflammatory medications are often used to alleviate pain, although they are not typically effective at addressing spinal compression, which is the cause of the pain.

Surgical treatments are more aggressive than medication or physical therapy, and in appropriate cases surgery may be the best way to achieve lessening of the symptoms of lumbar spinal stenosis and other spinal conditions. The principal goal of surgery to treat lumbar spinal stenosis is to decompress the central spinal canal and the neural foramina, creating more space and eliminating pressure on the spinal nerve roots. The most common surgery for treatment of lumbar spinal stenosis is direct decompression via a laminectomy and partial facetectomy. In this procedure, the patient is given a general anesthesia and an incision is made in the patient to access the spine. The lamina of one or more vertebrae may be partially or completely removed to create more space for the nerves. The success rate of decompressive laminectomy has been reported to be in excess of 65%. A significant reduction of the symptoms of lumbar spinal stenosis is also achieved in many of these cases.

The failures associated with a decompressive laminectomy may be related to postoperative iatrogenic spinal instability. To limit the effect of iatrogenic instability, fixation and fusion may also be performed in association with the decompression. In such a case, the intervertebral disc may be removed, and the adjacent vertebrae may be fused. A discectomy may also be performed to treat DDD and disc herniations. In such a case, a spinal fusion would be required to treat the resulting vertebral instability. Spinal fusion is also traditionally accepted as the standard surgical treatment for lumbar instability. However, spinal fusion sacrifices normal spinal motion and may result in increased surgical complications. It is also believed that fusion to treat various spinal conditions may increase the biomechanical stresses imposed on the adjacent segments. The resultant altered kinematics at the adjacent segments may lead to accelerated degeneration of these segments.

As an alternative or complement to the surgical treatments described above, an interspinous process device may be implanted between adjacent spinous processes of adjacent vertebrae. The purposes of these devices are to provide stabilization after decompression, to restore foraminal height, and to unload the facet joints. They also allow for the preservation of a range of motion in the adjacent vertebral segments, thus avoiding or limiting possible overloading and early degeneration of the adjacent segments as induced by fusion. The vertebrae may or may not be distracted before the device is implanted therebetween. An example of such a device is the interspinous prosthesis described in U.S. Pat. No. 6,626,944, the entire contents of which are expressly incorporated herein by reference. This device, commercially known as the DIAM® spinal stabilization system, is designed to restabilize the vertebral segments as a result of various surgical procedures or as a treatment of various spinal conditions. It limits extension and may act as a shock absorber, since it provides compressibility between the adjacent vertebrae, to decrease intradiscal pressure and reduce abnormal segmental motion and alignment. Due to its design, this device also stabilizes motion in flexion. Thus, the DIAM® device provides stability in all directions and maintains the desired separation between the vertebral segments all while allowing motion in the treated segment.

Although currently available interspinous process devices typically work for their intended purposes, they could be improved. For example, where the spacer portion of the implant is formed from a hard material to maintain distraction between adjacent vertebrae, point loading of the spinous process can occur due to the high concentration of stresses at the point where the hard material of the spacer contacts the spinous process. This may result in excessive subsidence of the spacer into the spinous process. In addition, if the spinous process is osteoporotic, there is a risk that the spinous process could fracture when the spine is in extension. In addition, because of the human anatomy and the complex biomechanics of the spine, some currently available interspinous process devices may not be easily implantable in certain locations in the spine.

The spine is divided into regions that include the cervical, thoracic, lumbar, and sacrococcygeal regions. The cervical region includes the top seven vertebrae identified as C1-C7. The thoracic region includes the next twelve vertebrae identified as T1-T12. The lumbar region includes five vertebrae L1-L5. The sacrococcygeal region includes five fused vertebrae comprising the sacrum. These five fused vertebrae are identified as the S1-S5 vertebrae. Four or five rudimentary members form the coccyx.

The sacrum is shaped like an inverted triangle with the base at the top. The sacrum acts as a wedge between the two iliac bones of the pelvis and transmits the axial loading forces of the spine to the pelvis and lower extremities. The sacrum is rotated anteriorly with the superior endplate of the first sacral vertebra angled from about 30 degrees to about 60 degrees in the horizontal plane. The S1 vertebra includes a spinous process aligned along a ridge called the medial sacral crest. However, the spinous process on the S1 vertebrae may not be well defined, or may be non-existent, and therefore may not be adequate for supporting an interspinous process device positioned between the L5 and S1 spinous processes.

Thus, a need exists for an interspinous process device that may be readily positioned between the L5 and S1 spinous processes without the reliance upon an S1 spinous process. Moreover, there is a need to provide an interspinous process device that can provide dynamic stabilization to the instrumented motion segment and not affect adjacent segment kinematics.

### SUMMARY

A spinal implant is described herein that is particularly adapted for placement between the spinous processes of the L5 vertebra and the S1 vertebra. The implant includes an upper saddle defined by a pair of sidewalls joined by a bottom wall. The upper saddle sidewalls may flare slightly outwardly away from the sagittal plane toward the top of the implant while the upper saddle bottom wall of the saddle may be concavely curved. In addition, the surfaces forming the upper saddle sidewalls and the upper saddle bottom wall extend in a direction, from the front of the implant to the rear of the implant, which is generally parallel to the sagittal plane. The implant also includes a lower saddle defined by a pair of sidewalls joined by a top wall. The lower saddle sidewalls flare outwardly away from the sagittal plane toward the bottom of the implant. In addition, the surfaces forming the lower saddle sidewalls extend in a direction, from the front of the implant to the rear of the implant, outwardly away from the sagittal plane. The lower saddle top wall may be concavely curved. In addition, the surface forming the lower saddle top wall extends in a direction, from the front of the implant to the rear of the implant, toward the top of the implant.

The spinal implant described herein has outer sidewalls that extend on either side of the implant from the upper portion of the implant to the lower portion of the implant. The outer sidewalls flare outwardly away from the sagittal plane from the upper portion of the implant to give the implant a generally triangular-like shape. The wider bottom portion of the implant allows two lower lobes to be defined along the bottom portion of the implant adjacent to either side of the lower saddle. The lower lobes each define a channel extending through the thickness of the implant. The channels allow a fixation device to extend therethrough to engage the pedicles of the inferior vertebra and thus fix the implant in the desired location. The channels may extend at an angle of about 60 degrees away from the sagittal plane toward the

The channels may be formed so that they allow the fixation device to extend through them at varying angles. This allows the surgeon to maneuver the fixation device along varying trajectories to engage the target pedicle at the desired location. This may be necessary given variations in the anatomy of different patients. In addition, a locking mechanism may be included in association with the channel to allow the trajectory of the fixation device to be fixed with respect to the spine to ensure that the implant maintains the desired orientation in the spine upon implantation. The locking mechanism may be a bushing disposed between the fixation device and the internal walls of the channel. In one orientation, the bushing may allow free rotation of the fixation device with respect to the channel and thus the implant. The shape of the bushing and the cross-section of the channel where the bushing is located are complementary to allow rotation of the bushing with respect to the channel and thus the implant. In another orientation, the bushing may lock the fixation device with respect to the channel and thus the implant. This locking feature may be achieved by using a fixation device that squeezes the bushing between the channel sidewall and the fixation device when the fixation device locks the implant to the spine.

### BRIEF DESCRIPTION OF THE DRAWINGS

FIG. 1 is a front perspective view of one embodiment of a lumbar-sacral implant allowing variable angle fixation;

FIG. 2 is a rear perspective view of the embodiment of the lumbar-sacral implant shown in FIG. 1;

FIG. 3 is a schematic, partial cross-sectional view of the left portion of the embodiment of the lumbar-sacral implant shown in FIG. 1 including a fixation device extending therethrough;

FIG. 4 is a side elevation view of the lumbar-sacral implant shown in FIG. 1 and including a fixation device extending therethrough;

FIG. 5A is a bottom cross-sectional view of another embodiment of the lumbar-sacral implant shown in FIG. 1 taken along line 5A-5A with a bushing disposed in each channel of the implant;

FIG. 5B is a schematic, partial cross-sectional view of the left portion of a further embodiment of the lumbar-sacral implant shown in FIG. 1 showing a fixation device with a further embodiment of the bushing and channel of the implant;

FIG. 5C is a schematic, partial cross-sectional view of a left portion of a still further embodiment of the lumbar-sacral implant shown in FIG. 1 showing a fixation device and a different configuration for the bushing and channel of the implant wherein the fixation device is not fully seated in the bushing;

FIG. 5D is a schematic, partial cross-sectional view of the left portion of the embodiment of the lumbar-sacral implant shown in FIG. 5C showing the fixation device locked with respect to the implant by the bushing disposed in the channel of the implant;

FIG. 5E is a schematic, partial cross-sectional view of a left portion of yet another embodiment of the lumbar-sacral implant shown in FIG. 1 showing a fixation device with another configuration for the bushing and channel of the implant wherein the fixation device is not fully seated in the bushing;

FIG. 5F is a schematic, partial cross-sectional view of the left portion of the embodiment of the lumbar-sacral implant shown in FIG. 5E showing the fixation device locked with respect to the implant by the bushing disposed in a channel of the implant;

FIG. 6 is a front elevation view of a lumbar-sacral implant similar to the one shown in FIG. 1 mounted on a spine but wherein the implant has a two-piece configuration;

FIG. 6A is a side, cross-sectional view of the lumbar-sacral implant shown in FIG. 6 illustrating the two-piece configuration; and

FIG. 7 is a side elevation view of the lumbar-sacral implant shown in FIG. 1 mounted on a spine.

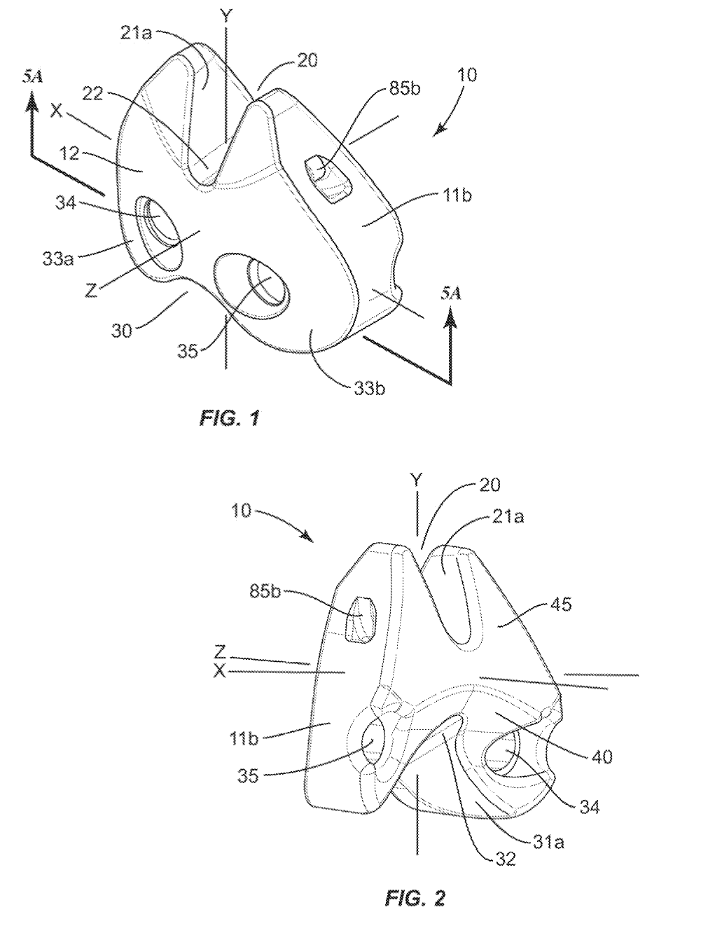

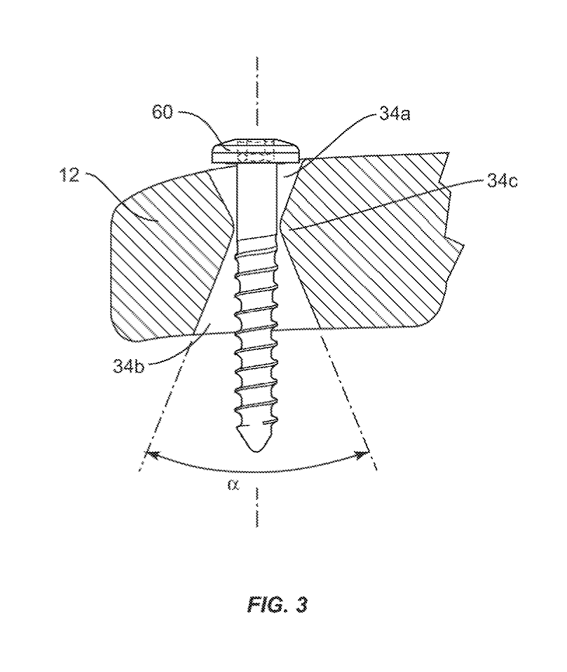

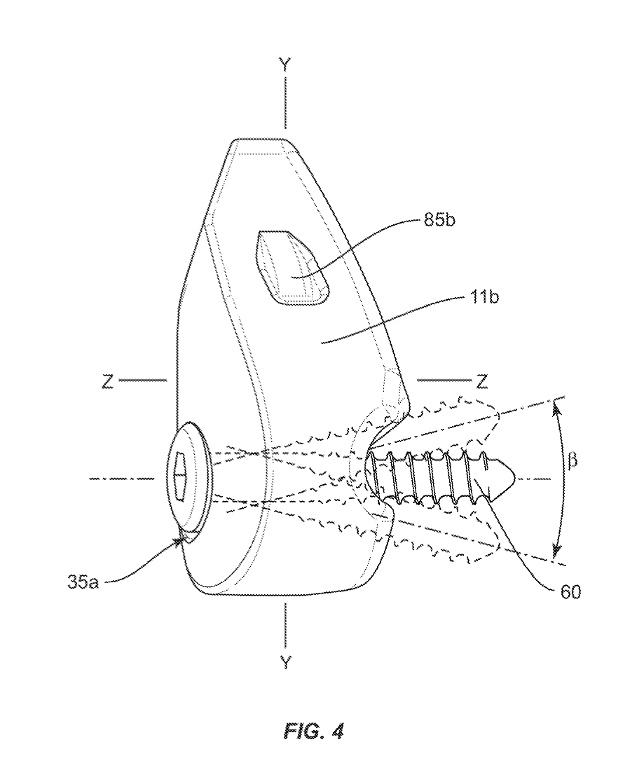

### DETAILED DESCRIPTION

As used in this specification and the appended claims, the singular forms “a,” “an” and “the” include plural referents unless the context clearly dictates otherwise. Thus, for example, the term “a member” is intended to mean a single member or a combination of members, and “a material” is intended to mean one or more materials, or a combination thereof. Furthermore, the words “proximal” and “distal” refer to directions closer to and away from, respectively, an operator (e.g., surgeon, physician, nurse, technician, etc.) who would insert the medical device into the patient, with the tip-end (i.e., distal end) of the device inserted inside a patient's body first. Thus, for example, the device end first inserted inside the patient's body would be the distal end of the device, while the device end last to enter the patient's body would be the proximal end of the device.

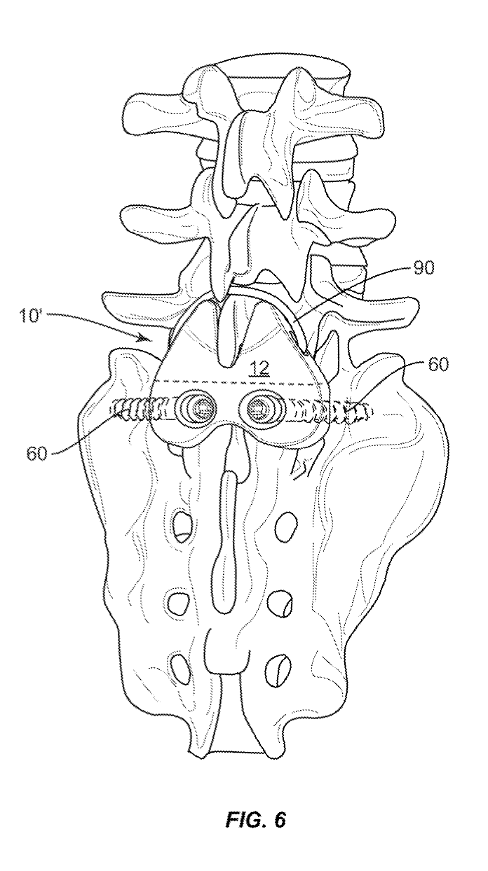

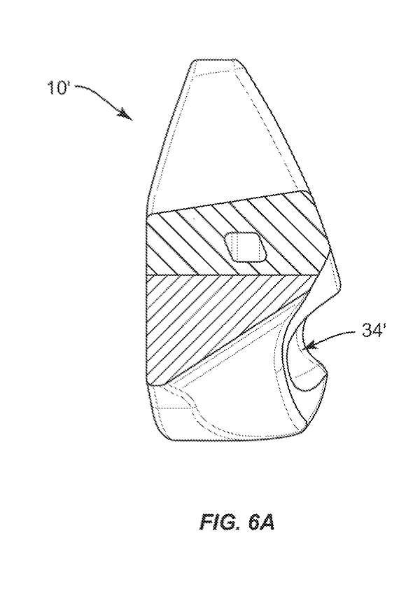

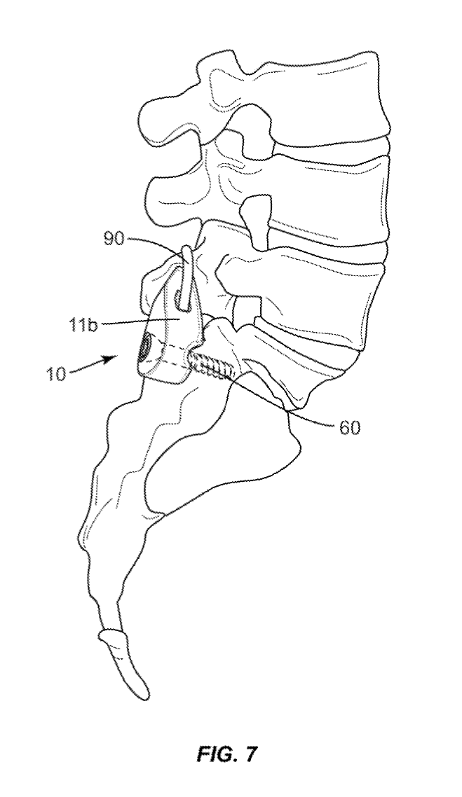

As used in this specification and the appended claims, the terms “up”, “upper”, “top”, “down”, “lower”, “bottom”, “front”, “back”, “rear”, “left”, “right”, “side”, “middle” and “center” refer to portions of or positions on the implant when the implant is oriented in its implanted position, such as shown in FIGS. 6 and 7.

As used in this specification and the appended claims, the term “axial plane” when used in connection with particular relationships between various parts of the implant means a plane that divides the implant into upper and lower parts. As shown in the FIGS., the axial plane is defined by the X axis and the Z axis. As used in this specification and the appended claims, the term “coronal plane” when used in connection with particular relationships between various parts of the implant means a plane that divides the implant into front and back parts. As shown in the FIGS., the coronal plane is defined by the X axis and the Y axis. As used in this specification and the appended claims, the term “sagittal plane” when used in connection with particular relationships between various parts of the implant means a plane that divides the implant into left and right parts. As shown in the FIGS., the sagittal plane is defined by the Y axis and the Z axis.

As used in this specification and the appended claims, the term “body” when used in connection with the location where the device of this invention is to be placed to treat lumbar spinal stenosis, or to teach or practice implantation methods for the device, means a mammalian body. For example, a body can be a patient's body, or a cadaver, or a portion of a patient's body or a portion of a cadaver. A “body” may also refer to a model of a mammalian body for teaching or training purposes.

As used in this specification and the appended claims, the term “parallel” describes a relationship, given normal manufacturing or measurement or similar tolerances, between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Thus, two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

As used in this specification and the appended claims, the terms “normal”, “perpendicular” and “orthogonal” describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal, perpendicular or orthogonal to a curved surface when the line and the curved surface intersect at an angle of approximately 90 degrees within a plane. Thus two geometric constructions are described herein as being “normal”, “perpendicular”, “orthogonal” or “substantially normal”, “substantially perpendicular”, “substantially orthogonal” to each other when they are nominally 90 degrees to each other, such as for example, when they are 90 degrees to each other within a tolerance. Such tolerances can include, for example, manufacturing tolerances, measurement tolerances or the like.

A spinal implant 10 is described herein that is particularly adapted for placement between the spinous processes of the L5 vertebra and the S1 vertebra. However, it is to be understood that even though the following description of implant 10 is provided with reference to the L5 spinous process and the S1 spinous process, implant 10 may be used between other adjacent spinous processes and the discussion of the L5 spinous process may be interpreted to include any superior spinous process and the S1 spinous process may be interpreted to include the adjacent inferior spinous process. Such an implant is disclosed, for example, in U.S. patent application Ser. No. (Docket No. P0039292.00) filed on even date herewith, entitled Viscoelastic Lumbar-Sacral Implant and naming Eric C. Lange et al. as inventors, the entire contents of which are hereby expressly incorporated herein by reference.

Implant 10 includes an upper saddle 20 defined by a pair of sidewalls 21a and 21b joined by a bottom wall 22. The upper saddle sidewalls may flare slightly outwardly away from the sagittal plane toward the top of implant 10 while upper saddle bottom wall 22 may be concavely curved. The left upper saddle sidewall 21a is shown in the FIGS. It is to be understood that the right upper saddle sidewall may be a mirror image of left upper saddle sidewall 21a. Implant 10 may have a variable radius, which may be from about 3.0 mm on the ventral face 12 to about 2.0 mm on the dorsal face 45. This allows implant 10 to engage the L5 spinous process, which is usually thicker at the base. Upper saddle 20 may be oriented at about a 10 degree angle in the sagittal plane. The angle could be as large as about 20 degrees. The surfaces forming the upper saddle sidewalls and upper saddle bottom wall 22 may be generally parallel to the sagittal plane. This configuration for upper saddle 20 allows upper saddle 20 to receive the spinous process of an L5 vertebra therein. The height of the upper saddle sidewalls may be chosen so that the upper saddle sidewalls prevent the upper portion of implant 10 from moving laterally out of engagement with the spinous process of the L5 vertebra. The upper saddle sidewalls may extend between ⅓ and ½ of the base of the spinous process so they engage the lamina by about 2 to 3 mm. The upper saddle sidewalls may or may not have a constant cross-section. This allows upper saddle 20 to accommodate the variable thickness of the spinous process. Implant 10 also includes a lower saddle 30 defined by a pair of sidewalls joined by a top wall 32. Left lower saddle sidewall 31a is shown in the FIGS. It is to be understood that the right lower saddle sidewall may be a mirror image of left lower saddle sidewall 31a. Lower saddle 30 has a configuration to provide clearance of implant 10 over the S1 spinous process. As such, lower saddle 30 would not engage the spinous process of the S1 vertebra. The lower saddle sidewalls flare outwardly away from the sagittal plane toward the bottom of implant 10.

Implant 10 may also define a curved passage that extends between the outer sidewalls of implant 10. The curve of this passage may be defined by a radius of curvature of about 20 millimeters where the openings to the passage, which are located on either side of implant 10, are closer to the top of implant 10 than the nadir of the passage. Right side opening 85b is shown in the FIGS. It is to be understood that a left opening also exists on the left side of implant 10. Other radii of curvature may also be used to define the passage. The nadir of the passage may be substantially aligned in the sagittal plane with the bottom most portion of upper saddle bottom wall 22 and the uppermost portion of lower saddle top wall 32. A tether 90 may extend through the passage. The curve of the passage facilitates tether 90 being threaded through the passage with a standard curved surgical needle.

The upper saddle sidewalls flare out and have a variable angle. The angle starts at about 40 degrees at the upper portion of upper saddle 20 and varies so that the angle is about 25 degrees at about the lowermost portion of upper saddle 20. The lower saddle sidewalls flare out and have a constant angle between about 25 degrees and about 35 degrees. Lower saddle top wall 32 may be concavely curved or may have another configuration that allows the lower portion of implant 10 to be fixed to the S1 pedicles and minimizes any interference between the S1 spinous process and the rear of implant 10. Lower saddle top wall 32 is inclined between about 30 degrees to about 35 degrees in the sagittal plane.

Implant 10 has outer sidewalls that extend on either side of implant 10 from the upper portion of implant 10 to the lower portion of implant 10. Right outer sidewall 11a is shown in the FIGS. It is to be understood that the left outer sidewall may be a mirror image of right outer sidewall 11a. The outer sidewalls flare outwardly away from the sagittal plane from the upper portion of implant 10 to give implant 10 a generally triangular-like shape when viewed from the front or the back. See e.g. FIG. 6. In addition, the overall shape of implant 10 transfers load from the L5 spinous process to the S1 pedicles instead of to the S1 spinous process or the S1 laminae. This is especially helpful where implant 10 is used in the L5-S1 level since the small size and shape of the S1 spinous process may not provide adequate support for an implant.

The front face 12 of implant 10 may have a curved profile that tapers from about 0 degrees along the middle of front face 12 to about 35 degrees adjacent to the outer sidewalls. Implant 10 may have a curvature radius of between about 20 mm and about 30 mm. The generally triangular shape, where the base is larger than the top, results in a constant pressure applied along the cross-sectional area of implant 10. The shape of implant 10 also provides a better fit in the L5/S1 space and therefore offers stability for implant 10. The rear of implant 10 has a stepped configuration and includes a shelf 40 separating the rear of implant 10 into an upper portion and a lower portion. Shelf 40 may be curved and is located so it is generally aligned with or above channels 34 and 35. Shelf 40 acts as a transition between the upper and lower portions of the rear of implant 10 and ensures that implant 10 will fit properly in the patient's anatomy. The upper rear portion of implant 10 is defined by the rear wall 45, which flares outwardly from the top of implant 10. Rear wall 45 is curved such that it does not compete for engagement with upper saddle 20 but rather allows implant 10 to rest freely on the L5 lamina. This allows for easy implantation on the L5 level. The thickness of implant 10 gradually increases from the top of implant 10 to shelf 40. This taper may be between about 30 degrees and about 50 degrees. The bottom rear portion of implant 10 has a thinner profile and provides clearance so that lower saddle 30 does not engage the inferior spinous process. This results in practically no load being transferred from implant 10 to the inferior spinous process. Indeed, lower saddle 30 may be configured such that it is spaced from the inferior spinous process when implant 10 is implanted in the patient.

The wider bottom portion of implant 10 allows two lower lobes 33a and 33b to be defined along the bottom portion of implant 10 adjacent to either side of lower saddle 30 and provides an area through which implant 10 may be fixed to the spine. Each lower lobe 33a and 33b defines a channel 34 and 35, respectively, extending through implant 10. The wider bottom portion of implant 10, and indeed the overall configuration of implant 10, also allows implant 10 to withstand higher forces being placed on it and helps to ensure that the compression forces placed on implant 10 are evenly distributed throughout the body of implant 10.

Each of channels 34 and 35 allows a fixation device 60, such as a cortical screw or similar device, to extend through each of them to engage the pedicles of the inferior vertebra and thus fix implant 10 in the desired location on the spine. The internal diameter of channels 34 and 35, at a minimum, should be sufficient to allow passage of fixation device 60 therethrough. In addition, each channel 34 and 35 may each have a converging tapered portion 34a and a diverging tapered portion 34b that are joined at a narrowed waist portion 34c along a medial portion of each channel. See FIG. 3. Although the right side of implant 10 is not shown, it is to be understood that right channel 35 has the same general configuration as left channel 34. Waist portion 34c acts as a pivot location for fixation device 60. The combination of converging portion 34a adjacent to the front of implant 10, narrowed waist portion 34c and diverging portion 34b adjacent to the rear of implant 10 allows each fixation device 60 to extend through each channel 34 and 35 and allows the surgeon to maneuver fixation device 60 along varying trajectories to engage the target pedicle at the desired location. For example, converging portion 34a and diverging portion 34b may be configured to allow fixation device to extend along a lateral, i.e. side to side, angle α, which may be between about 30 degrees and about 60 degrees, and along a superior-inferior, i.e. up and down, angle β, which may be between about 5 degrees and about 10 degrees. Thus in one embodiment, converging portion 34a and diverging portion 34b may have an asymmetrical cone-like cross-section where the angle of the side walls taper at an angle of between about 30 degrees and about 60 degrees and the angle of the top and bottom walls taper at an angle of between about 5 degrees and about 10 degrees. These angles of course would smoothly transition together around the circumference of these portions of channels 34 and 35. Alternatively, the cross-sections of converging portion 34a and diverging portion 34b could be symmetrical and taper at an appropriate angle between about 15 degrees and about 75 degrees.

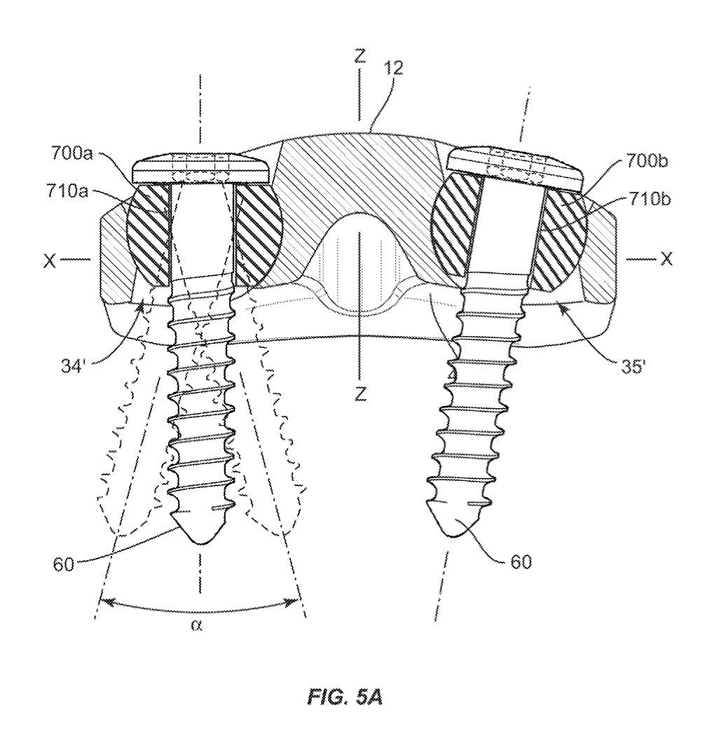

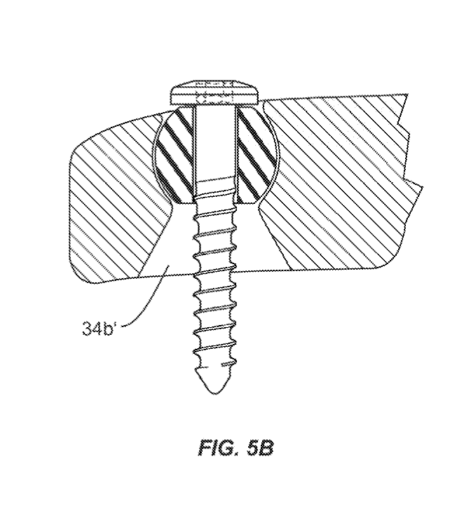

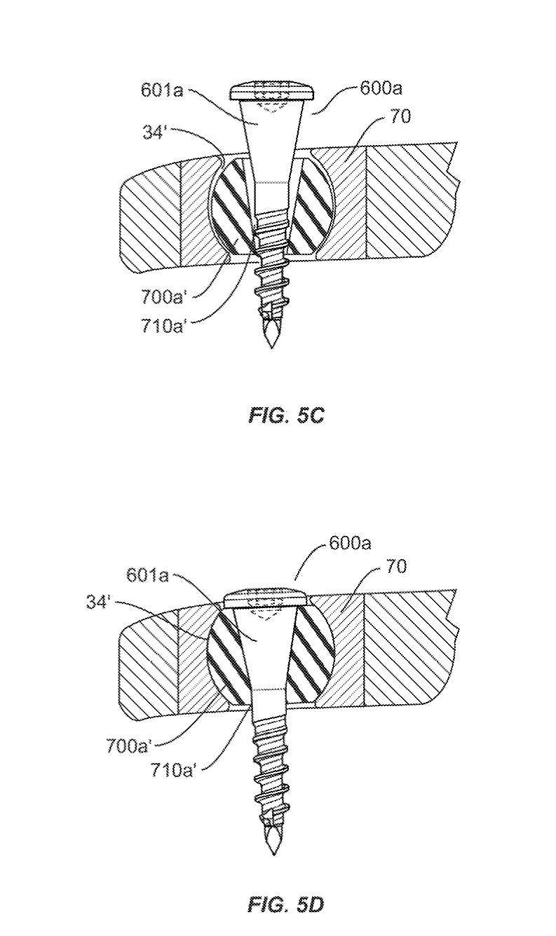

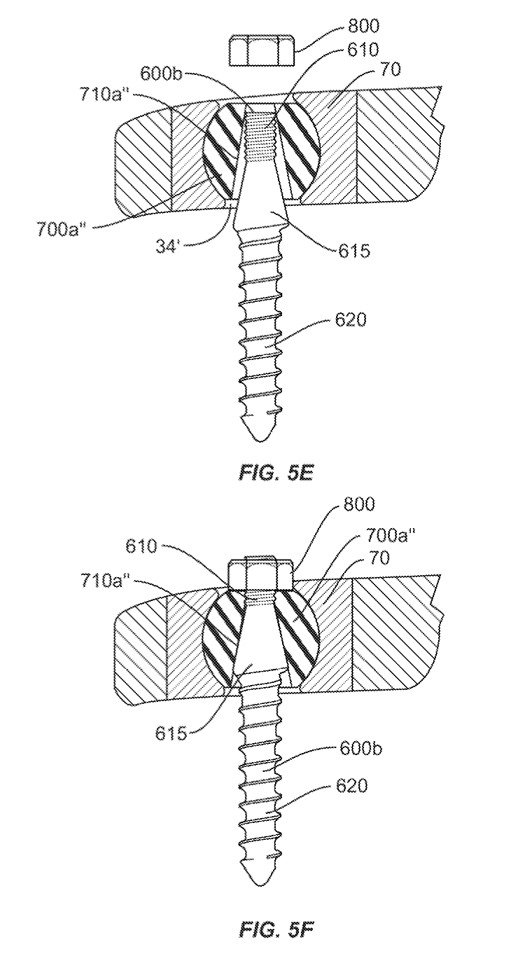

As shown in FIGS. 5A through 5F, bushings 700a and 700b may be disposed in respective channels 34′ and 35′ through which respective fixation devices 60 may extend. Bushings 700a and 700b may be formed of any biocompatible material and may be relatively rigid, such as PEEK or titanium. Bushings 700a and 700b may be generally spherical, i.e. have a generally circular cross-section, with a passage 710a and 710b therethrough, to allow fixation device 60 to pass through bushings 700a and 700b. Similarly, channels 34′ and 35′ may have a generally spherical cross-section in the area where bushings 700a and 700b are to be located to hold bushings 700a and 700b in place. The spherical configuration of bushings 700a and 700b and channels 34′ and 35′ allow bushings 700a and 700b to freely rotate within channels 34′ and 35′. This in turn allows fixation device 60 to be rotated within channels 34′ and 35′ with some degree of control because bushings 700a and 700b provide a bearing surface in the gap between the respective fixation device 60 and the inner surface of the respective channel 34′ and 35′.

If desired, the rear portion of channels 34′ and 35′ may flare outwardly so that the cross-section of the channels flare toward a larger diameter at the rear of the implant. See element 34b′ in FIG. 5B. It is to be understood that the right channel may have the same general configuration. In other words, the diameter of channels 34′ and 35′ increases from the spherical portions to the rear of the implant. This configuration allows room in channels 34′ and 35′ for fixation device 60 to have wide latitude in rotating within these channels. The particular angle of the taper for this portion of channels 34′ and 35′ may be as described in connection with the embodiment shown in FIG. 3.

In a further embodiment, the fixation device used with the implant described herein may have a tapered shaft portion 601a that tapers to a smaller diameter toward its distal end. Bushing 700a′ may also define a tapered passage therethrough to mate with tapered shaft portion 601a. See FIGS. 5C and 5D. Although only the left portion of the implant is shown, it is to be understood that the right channel, right bushing and right fixation device have the same configuration as the left channel, left bushing and left fixation device. During implantation, the surgeon will locate implant 10 in the interspinous space between the L5 vertebra and the S1 vertebra. Once the surgeon locates implant 10 in the desired location, fixation device 600a may be inserted through channels 34′ and 35′. The trajectory of fixation device 600a may be adjusted during this phase of the operation to ensure that the distal end of fixation device engages the desired location of the target pedicle. Fixation device 600a is able to rotate with respect to the implant in this orientation because bushing 700a has not been squeezed between the channel sidewalls and the fixation device since the narrower portion of the shaft of fixation device 600a is in engagement with passage 710a. See FIG. 5C. Once fixation device 600a is properly aligned, it is driven into the pedicle. Continued movement of fixation device 600a into the pedicle forces tapered portion 601a into passage 710a′ so that tapered portion 601a fully engages bushing 700a thus squeezing bushing 700a within channel 34′ by fixation device 600c. See FIG. 5D. In this configuration, fixation device 600a is locked in position with respect to the implant. As shown in FIGS. 5C and 5D, the implant may include a grommet 70 disposed about the channels. Such a grommet 70 may have different hardness characteristics to ensure that the bushings are locked in place with respect to the implant. Alternatively, the bottom portion of the implant may be formed from a separate material that is more rigid than the top of the implant. See FIGS. 6 and 6A.

Another embodiment of a fixation device that may be used with the implant described herein is shown in FIGS. 5E and 5F. Fixation device 600b may have a tapered shaft portion and a nut to force the tapered shaft portion into cooperation with bushing 700a″ and the channel to lock the fixation device with respect to the implant. Although only the left portion of the implant is shown in the FIGS., it is to be understood that the right channel, right bushing and right fixation device have the same general configuration as the left channel, left bushing and left fixation device. As shown in FIGS. 5E and 5F, fixation device 600b includes a tapered portion 615 located between a proximal portion 610 and a distal portion 620. The taper of tapered portion 615 is reverse to the taper shown in FIGS. 5C and 5D such that the taper of tapered portion 615 increases in diameter in the distal direction. Bushing 700a″ also defines a passage 710a″ that has a taper that increases toward the rear of each bushing. Proximal portion 610 is threaded to receive a nut 800 thereon. Distal portion 620 is threaded so fixation device 600b can be driven into a pedicle. During implantation, the surgeon inserts fixation device 600b into the pedicles first. Once, the fixation devices are properly located, the surgeon may place implant 10 in the interspinous space between the L5 vertebra and the S1 vertebra over the fixation devices through the channels. When the implant is partially placed over the fixation devices, the bushings are able to rotate with respect to the implant in this orientation because the bushings have not been squeezed within the channel by the fixation device since the larger tapered portion of the shaft of the fixation device is not in engagement with the bushing passage. See FIG. 5E. Once the implant is properly aligned, it is driven completely over the fixation devices so that the larger tapered portions fully engage the bushings thus squeezing the bushings in the channels. See FIG. 5F. Nut 800 is disposed over proximal portion 610, threaded over the threads and rotated to engage the front of bushing 700a″. In this configuration, fixation device 600b is locked in position with respect to the implant.

While various embodiments of the flexible interspinous process device and delivery system have been described above, it should be understood that they have been presented by way of example only, and not limitation. Many modifications and variations will be apparent to the practitioner skilled in the art. The foregoing description of the flexible interspinous process device and delivery device is not intended to be exhaustive or to limit the scope of the invention. It is intended that the scope of the invention be defined by the following claims and their equivalents.

What is claimed is:

1. A device, comprising:
a front face;
a rear face;
an upper body portion defining an upper saddle;
a lower body portion defining a lower saddle; the upper body portion separated from the lower body portion;
the lower body portion including a left lower lobe and a right lower lobe, each lobe being adjacent to an opposite side of the lower saddle;
a left channel extending through the device in the left lower lobe and a right channel extending through the device in the right lower lobe;
a left bushing disposed in the left channel and a right bushing disposed in the right channel; and
a left fixation device adapted to extend through the left channel and the left bushing and a right fixation device adapted to extend through the right channel and the right bushing wherein the left fixation device cooperates with the left channel and the left bushing so that an angular orientation of the left fixation device with respect to the implant may be varied and wherein the right fixation device cooperates with the right channel and the right bushing so that an angular orientation of the right fixation device with respect to the implant may be varied.

2. The device of claim 1 wherein the left bushing and the right bushing each has a generally spherical configuration and the left bushing defines a left passage extending therethrough and the right bushing defines a right passage extending therethrough.

3. The device of claim 2 wherein the left bushing and the right bushing are flexible.

4. The device of claim 2 wherein the left bushing and the right bushing are rigid.

5. The device of claim 2 wherein the left fixation device includes a first tapered portion and the right fixation device includes a second tapered portion.

6. The device of claim 2 wherein the left channel includes a first generally spherical cross-sectional portion and the left bushing is disposed in the first generally spherical cross-sectional portion and the right channel includes a second generally spherical cross-sectional portion and the right bushing is disposed in the second generally spherical cross-sectional portion.

7. The device of claim 6 wherein the left channel includes a first tapered portion adjacent to the first generally cross-sectional portion wherein the first tapered portion increases in diameter toward a rear portion of the device and the right channel includes a second tapered portion adjacent to the second generally cross-sectional portion wherein the second tapered portion increases in diameter toward the rear portion of the device.

8. The device of claim 6 wherein the left bushing is rotatably disposed in the first generally spherical cross-sectional portion and the right bushing is rotatably disposed in the second spherical cross-sectional portion.

9. The device of claim 8 wherein the left fixation device cooperates with the left channel and the left bushing so that an angular orientation of the left fixation device may also be fixed with respect to the implant and wherein the right fixation device cooperates with the right channel and the right bushing so that an angular orientation of the right fixation device may also be fixed with respect to the implant.

10. A device, comprising:
a front face;
a rear face;
an upper body portion defining an upper saddle;
a lower body portion defining a lower saddle;
a first channel extending through the device wherein the channel has a first portion that has a varying cross-section that tapers to a waist portion adjacent to a medial portion of the channel; and
a fixation device extending through the passage and the first channel and pivotable about the waist portion such that the trajectory of the fixation device with respect to the device may be adjusted.

11. The device of claim 10 where the channel further includes a second tapered portion having a varying cross-section that diverges from the waist portion to the rear face.

12. The device of claim 10 wherein the first channel is configured so that the trajectory of the fixation device through the first channel may be adjusted about an angle of between about 15 degrees and about 75 degrees around the circumference of the first channel.

13. The device of claim 12 wherein the first channel is configured so that the trajectory of the fixation device through the first channel may be adjusted side to side about an angle between about 30 degrees and about 60 degrees.

14. The device of claim 12 wherein the first channel is configured so that the trajectory of the fixation device through the first channel may be adjusted up and down about an angle between about 5 degrees and about 10 degrees.

15. A method of implanting a device, comprising:
locating the device in a desired location;
extending a fixation element through the device;
changing the trajectory of the fixation element with respect to the device while the fixation element is extending through the device;
aligning the fixation element with a desired trajectory; and
locking the trajectory of the fixation element with respect to the device.

16. The method of claim 15 wherein the device includes a channel and a bushing disposed in the channel and the changing the trajectory step includes rotating the bushing with respect to the channel.

17. The method of claim 15 wherein the locking the trajectory step includes changing the configuration of the bushing.

18. The method of claim 17 wherein the locking the trajectory step includes compressing the bushing.

19. The method of claim 15 wherein the aligning step is performed before the extending step.
